(19) United States
(12) Patent Application Publication
(10) Pub. No.: US 2004/0138528 A1
(43) Pub. Date: Jul. 15, 2004

(54) Surgical instrument

(76) Inventors: Jorn Richter, Munster (DE); Andreas Bracke, Bochum (DE); Dietrich H W Gronemeyer, Bochum (DE); Jurgen Speder, Bochum (DE); Manfred Weck, Aachen (DE); Stefan Fischer, Eindhoven (NL); Sven Carsten Lange, Aachen (DE); Benno Brocher, Roedermark (DE); Florian Schmidt, Stuttgart (DE); Daniel E Spielberg, Altdorf (DE)

Correspondence Address: RALPH W. SELITTO, JR., C/O MCCARTER & ENGLISH, LLP, GATEWAY CENTER FOUR, 100 MULBERRY STREET, NEWARK, NJ, 07102, US

(21) Appl. No.: 10/398,926

(22) PCT Filed: Oct. 11, 2001
(86) PCT No.: PCT/DE01/03864

(30) Foreign Application Priority Data
Oct. 12, 2000 (DE) 100 50 648.8

(51) Int. Cl. A61B 1/00

(52) U.S. Cl. 600/134

(57) ABSTRACT

A surgical instrument is disclosed, preferably for the minimally invasive intervention in human or animal tissues comprising a, for example, needle-shaped base body (3) with at least one passage formed from a hollow fiber (2). The at least one hollow fiber (2) is surrounded by a matrix (5) of a fibre composite material. The use of hollow fiber (2) permits a simple production in particular of instruments with a number of passages. The use of more than one hollow fiber (2) within the base body (3) increases the application possibilities, for example, simultaneous monitoring and treatment of the tissue.

This invention relates to a surgical instrument, comprising a base body of a fibrous composite having at least one lead-through, which extends from a posterior end of the base body to an anterior end.

Surgical instruments of the aforementioned type are preferred for minimally invasive surgical procedures. Such a surgical instrument is known from International Patent WO 97/07746, which relates to a needle having a passage for fluids to flow through or for the use of other instruments, e.g., optical fibers, catheters, trocars, etc. The needle is pointed at its distal end so that it can enter into the tissue to be treated or examined without a prior separate incision.

In minimally invasive procedures, it is usually important for the navigation of the surgical instrument to be supported by imaging during the treatment in order to ascertain the position of the instrument in the tissue. Nuclear magnetic tomography, also known as magnetic resonance imaging (MRI) is recommended for this purpose, along with such other tomographic methods as computer tomography (CT scan). However, surgical instruments having a magnetic susceptibility which differs greatly from that of the tissue to be treated or examined can interfere with or entirely prevent correct imaging of the region to be imaged due to artifacts within the image, because sudden changes in magnetic susceptibility result in distortion in the uniform magnetic field used in MRI. Therefore, the state of the art which is cited has proposed that the surgical needle be made of a nonmetallic material, preferably a fibrous composite comprising carbon fibers. A “pultrusion” method is proposed for the production of this needle; in this method, the base body is placed over a core in a drawing press. The core is then removed to produce the lead-through. As an alternative, the prepared base body may also be wrapped around the core.

The method proposed above for producing the known needles is complicated in particular because of the need for a core and for removing the core.

It is the object of the present invention to provide a surgical instrument of the type defined above, which can be produced in a simplified manner in comparison with the state of the art.

This object is achieved with a surgical instrument of the type defined in the preamble due to the fact that the at least one tubular hollow space is formed by a hollow fiber embedded in the base body.

When using a hollow fiber, it may be embedded in the base body made of a fibrous composite. This eliminates the need for removing a core.

In addition, it may be advantageous to design the surgical instrument so that the hollow fiber is a hollow glass fiber. Hollow glass fibers have the advantage that they can be used to provide a passage for function elements, e.g., optical fibers, and also for conducting light. For example, when an endoscope is passed through such a hollow fiber, the light of a light source needed for the endoscope may also at the same time be conducted through the hollow glass fiber to the site observed.

The surgical instrument according to this invention may also be designed so that the base body contains carbon fibers embedded in a matrix.

In addition, the surgical instrument according to this invention may be designed so that the matrix is formed from a thermosetting plastic, e.g., an epoxy resin.

The surgical instrument according to this invention may also be designed so that the matrix is formed from a plastic which is biodegradable in the human or animal body.

In addition, plastic fibers, e.g., those made of Aramid, metal fibers, ceramic fibers, carbon fibers and natural fibers, e.g., fibers made of hemp may also be used for the fibers of the base body as well as the hollow fibers. Ceramic fibers, carbon fibers, plastic fibers and natural fibers have advantageous magnetic properties for the use of MRI for imaging. Natural fibers are also advantageous because of their biodegradability.

Depending on the application, elastomers, ceramics, glass, carbon and metal may be advantageous matrix materials.

In addition, it may be advantageous to design the surgical instrument according to this invention so that at least two hollow fibers running essentially parallel to one another are provided. This permits the simultaneous use of several measures. For example, a hollow fiber may be used for endoscopy and also a second hollow fiber may be used for suction removal of fluid, to administer medication or to allow another function element to pass through. Function elements may include, for example, optical fibers, current-carrying lines or surgical tools, e.g., for a biopsy.

The surgical instrument according to this invention may also be designed so that a current-carrying line which runs essentially parallel to the at least one hollow fiber is provided, and the current-carrying line is electrically insulated from the outside space of the surgical instrument in the radial direction. Certain methods of treatment, such as cauterizing tissue, require the use of an electric current, which may be made available in this way. The current-carrying line may be insulated, e.g., by means of sheathing by a hollow glass fiber. It may be advantageous for the current-carrying lines to be made of carbon fibers.

The surgical instrument according to this invention may also be designed so that the base body is flexible in at least a distal end area. Flexibility may be advantageous in particular when the surgical instrument is to be inserted into pre-existing body cavities, and in doing so, should follow a path defined by tissue, e.g., in the intestine. The base body of the surgical instrument according to this invention may of course also be rigid.

In addition, it may be advantageous to design the surgical instrument according to this invention so that the base body has a sharpened distal end with which it is possible to produce an opening which permits access to human or animal tissue. This would eliminate the need for a separate incision.

It may also be advantageous to design the surgical instrument according to this invention, so that the base body is coated in at least a distal region on its circumference. A coating may be impart stability to the tip, in particular to prevent the loss of fiber material or matrix material into the tissue. Ceramic materials or wear-resistant plastics in particular are suitable for this coating. Metals and metal alloys such as brass may also be used.

The surgical instrument according to this invention may also be designed so that the fibers present in a distal region of the base body are stabilized with respect to interaction with human or animal tissue. In the case of carbon fibers, this stabilization may be accomplished, e.g., by immersing the tip of the surgical instrument in liquid silicon, thereby ceramizing the tips of the fiber to form SiC.

The surgical instrument according to this invention may also be designed so that a connecting element for connecting to an operating device is provided on the proximal end of the base body. An operating device is used, first of all, for guiding the surgical instrument. Secondly, function elements, e.g., endoscopes, optical fibers, gripper elements, lasers, current-carrying lines, etc. may be supplied to the surgical instrument via the operating device and also controlled by it. Furthermore, it is possible to add substances, e.g., rinsing fluid, medication or tissue, via the operating device or to remove them via the operating device. The various measures may also be implemented concurrently, which hardly appears feasible with the state of the art described in the preamble.

Finally, the surgical instrument according to this invention may also be designed so that a function element which can be connected to the operating device via the connecting element is provided in the hollow fibers or in at least one of the hollow fibers. For example, this may be an endoscope, which must then need no longer be inserted separately into a hollow fiber of the surgical instrument after being connected to the operating device.

The function element may also be understood to be a sealing element which seals the hollow fiber on the distal end. Such a sealing element may, for example, prevent the penetration of tissue on insertion of the surgical instrument or the admission of other substances into a hollow fiber not intended for this purpose. The closing element may be a cylindrical pin having a diameter which adequately fills up the corresponding hollow fiber.

An advantageous embodiment of the surgical instrument according to this invention is described below on the basis of figures.

They show schematically:

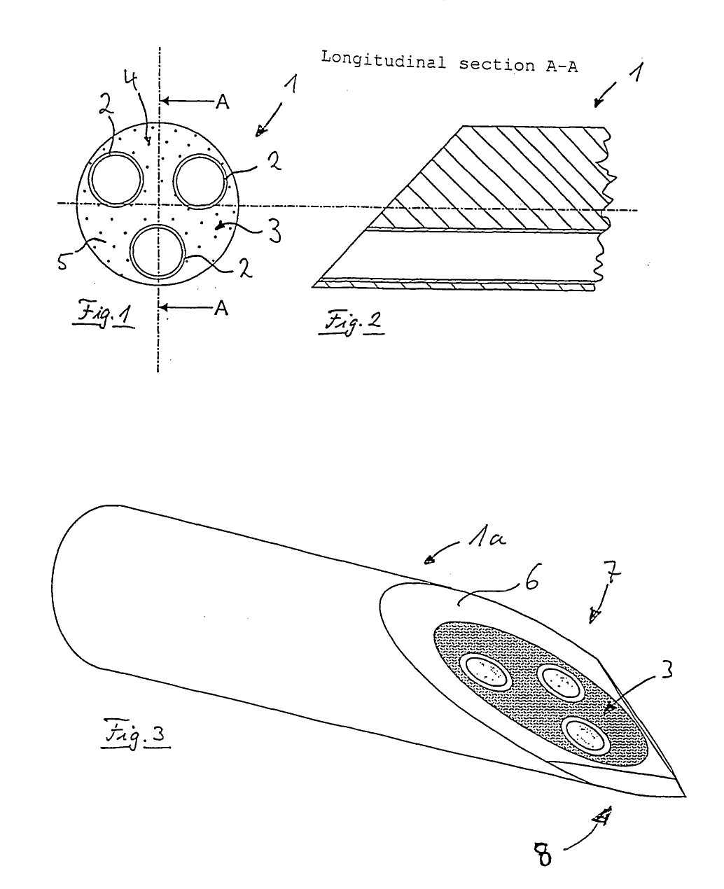

FIG. 1: a surgical instrument in the form of a needle shown in cross section;

FIG. 2: a portion of the needle according to FIG. 1 in a lateral longitudinal section;

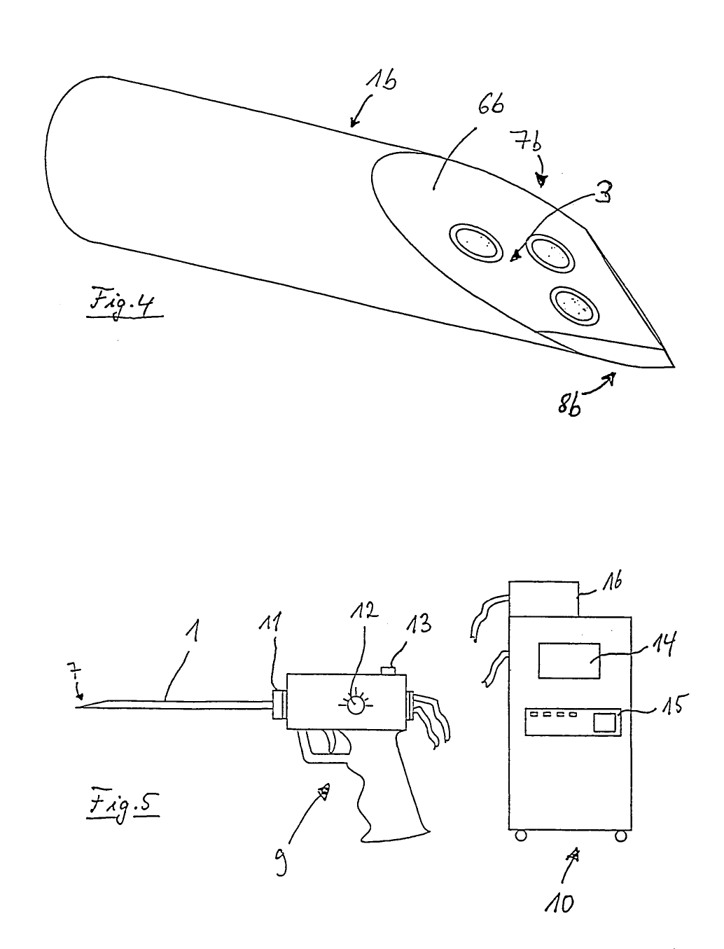

FIG. 3: the tip of a needle cut off after coating;

FIG. 4: the tip of a needle coated after being cut off;

FIG. 5: a system comprising a needle, an operating device and a basic module.

A surgical needle 1, which is shown schematically in a cross-sectional view in FIG. 1, has three hollow fibers 2 made of glass. The three hollow fibers 2 are surrounded by a base body 3 made of a fibrous composite. The base body 3 comprises carbon fibers 4, which are arranged essentially in parallel with the hollow fibers 2 and are embedded in a matrix 5 of epoxy resin.

FIG. 2 shows the needle 1 in a longitudinal section A-A at its distal end. One of the hollow fibers 2 is visible here. The needle 1 is pointed and sharpened at its distal end, so that it can penetrate into human or animal tissue without requiring a separate incision in advance. When inserting the needle it is possible to fill up the hollow fiber 2 with a tubular closing element (not shown here) to prevent unwanted admission of tissue into the hollow fibers 2 in the movement of needle 1 through the tissue.

FIG. 3 shows the tip of a needle 1a, which is coated on its lateral cylindrical surface. The layer 6a of ceramic was applied to a base body strand before needle 1a was cut off from this strand. The tip 7a of the needle 1a was thus prepared only after the coating. The sharpened cutting area 8a of the tip 7a consists entirely of the ceramic layer 6a to prevent the fibers 4 and/or the matrix material 5 of the base body 3 from remaining in the tissue to be examined or treated.

FIG. 4 shows the tip 7b of a needle 1b, which has been coated only after the base body 3 was cut off from the base body strand (not shown here). This procedure is somewhat more complicated, but it has the advantage that the complete tip 7b is also provided with a ceramic layer 6b. The cutting area 8b is sharpened after coating.

FIG. 5 shows schematically a complete multifunction system comprising the needle 1, an operating device 9 and a basic module 10. The versatile application possibilities of the system are explained below. The needle 1 is connected to the operating device 10 by means of a bayonet closure 11, which is sealed to prevent loss of liquid. By means of the operating device 10, function modules, e.g., glass fiber bundles for endoscopy, means for taking samples of tissue or current-carrying lines, etc. (not shown separately here) can be introduced into the hollow fibers, and their position can be adjusted and monitored within hollow fibers 2. For example, FIG. 4 shows a focus-adjusting screw 12 for the movement of glass fiber bundles for endoscopy as an example; it is used, first of all, for inserting the glass fiber bundle and at the same time aligning the focal point of the respective lens at a certain object within the tissue. Information obtained by means of optical fiber bundles can be transmitted to an image analyzer, which is provided in the basic module 10 via an actual intermediate image in the operating device 9. In this way, an optical fiber need not lead all the way from the tip 7 of the needle to the image analyzer. By means of a fiber inserted into the hollow fibers 2, laser light may also be introduced for ablation of tissue. However, laser light could also be guided within a transparent fluid which is conveyed through one or more of the hollow fibers 2 to the desired site in the tissue.

To be able to supply substances such as rinsing fluid or medication to the tissue through one of the hollow fibers 2, there is a Luer lock 13 on the operating device 19 to which inlet lines (not shown here) can be connected. The presence of a plurality of hollow fibers 2 in the needle 1 has the advantage in particular that different functions can be fulfilled by the needle 1 simultaneously, e.g., an observation function and a rinsing function, which are accommodated in separate hollow fibers.

The operating device 9 may be controlled manually on the operating device 9 itself or electronically via the basic module 10. Basic module 10 is therefore equipped with a monitor 14 and a control unit 15. Basic module 10 may also have a laser source 16 or other light sources (not shown here), e.g., for endoscopy.

List of Reference Notation

| | |
|---|---|
| 1 | needle |
| 2 | hollow fiber |
| 3 | base body |
| 4 | carbon fiber |
| 5 | matrix |
| 6 | ceramic layer |
| 7 | tip |
| 8 | cutting area |
| 9 | operating device |
| 10 | basic module |
| 11 | bayonet closure |
| 12 | focus-adjusting screw |
| 13 | Luer lock connection |
| 14 | monitor |
| 15 | control unit |
| 16 | laser source |

1. A surgical instrument, comprising a base body (3) made of a bonded fiber material having at least one lead-through extending from a proximal end to a distal end of the base body (3), characterized in that the at least one lead-through is formed by a hollow fiber (2) surrounded by the base body (3), whereby the wall thickness of the base body (3) is several times greater than that of the hollow fiber (2), as seen in at least most radial directions from the central longitudinal axis of the at least one lead-through.

2. The surgical instrument according to claim 1, characterized in that the hollow fiber (2) is a hollow glass fiber.

3. The surgical instrument according to claim 1 or 2, characterized in that the base body (3) contains carbon fibers (4) embedded in a matrix (5).

4. The surgical instrument according to claim 3, characterized in that the matrix (5) formed from a thermosetting plastic, e.g., an epoxy resin.

5. The surgical instrument according to claim 3, characterized in that the matrix (5) is formed from a thermoplastic.

6. The surgical instrument according to claim 3, characterized in that the matrix (5) is formed from a plastic which is biodegradable in the human or animal body.

7. The surgical instrument according to one of the preceding claims, characterized in that at least two hollow fibers (2) running essentially parallel to one another are provided.

8. The surgical instrument according to one of the preceding claims, characterized in that a current-carrying line which runs essentially parallel to the at least one hollow fiber (2) is provided, and the current-carrying line is electrically insulated with respect to the outside space of the surgical instrument in the radial direction.
